(19) United States
(12) Patent Application Publication
(10) Pub. No.: US 2005/0089861 A1
(43) Pub. Date: Apr. 28, 2005

(54) Method of lancing skin for the extraction of blood

(76) Inventor: John J Allen, Mendota Heights, MN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/495,408

(22) PCT Filed: Oct. 29, 2003
(86) PCT No.: PCT/US03/34456

Related U.S. Application Data
(60) Provisional application No. 60/422,228, filed on Oct. 30, 2002.

(51) Int. Cl. C12Q 1/68; A61M 31/00

(52) U.S. Cl. 435/6; 604/500

(57) ABSTRACT

The present invention relates to a method of lancing skin through an outer surface of said skin to obtain a sample of blood, the method providing a lancing instrument having a sharpened end and a channel extending from adjacent said sharpened end to a sensor attached to a proximal end of said lancing instrument, forcing said sharpened tip into said skin to a first predetermined depth below said outer surface, wherein said sharpened tip creates an incision in said skin surface; then completely withdrawing said sharpened tip from the incision, and drawing blood through said channel to the sensor.

### BACKGROUND OF THE INVENTION

1. Field of the Invention

The present invention relates, in general, to a method of lancing skin and, more particularly, to an improved method wherein the lancing element is removed after creating an incision and then blood is channeled up the lancing element to a sensor element located at a proximal end of the lancing element.

2. Background of the Invention

In in-situ testing of blood glucose, a glucose meter is placed against the skin and blood is sampled and measured without moving the meter. In one method of in-situ testing, a glucose sensor strip is combined with a lancing element positioned at a distal end thereof, the glucose sensor strip is then positioned in a meter adapted to launch the strip and lancing element combination toward the skin where the lancing element forms an incision. Blood or other bodily fluids such as, for example, interstitial fluid, may then be extracted from the incision and moved to the glucose sensor strip where it can be measured using, for example, an electrochemical process.

When lancing skin using an in-situ test strip, it is desirable to ensure that blood be transferred efficiently from the incision to the test strip, using as little blood as possible. Efficient transfer of blood from the incision means that more of the blood is actually used to test for analyte (e.g. glucose) levels, reducing the total blood required and, therefore, the incision size required for the test. Smaller incisions are particularly desirable because, in general, it is desirable to reduce the pain experienced by the user. Further, smaller incisions generally heal faster and are not as likely to re-open once healed.

Thus, when using an in-situ test, it is desirable to create an incision which is very small while maximizing the amount of blood generated by that incision. A number of factors influence the amount of blood generated by a particular incision. Many of those factors cannot be controlled. One of the factors which reduces the amount of blood available at a particular incision is the tendency of the incision to seal around the lancing element if the lancing element is left in the wound.

It would, therefore, be advantageous to develop a method of lancing which increases the amount of blood available for testing at a particular incision site. It would further be advantageous to develop a method of lancing which increases the amount of blood available for lancing by preventing the wound from resealing during the testing process. It would further be advantageous to develop a method of lancing which increases the amount of blood available for lancing by preventing the wound from sealing around the lancing element during the testing process.

### SUMMARY OF THE INVENTION

In a method according to the present invention, a lancing tip is first inserted and then is retracted completely out of the lance wound site. The lancing tip is then positioned adjacent the wound opening and blood is channeled to a test strip integrated with the lance.

In a method of lancing skin according to the present invention, the lancing element is inserted through an outer surface of the skin to obtain a sample of blood. In one embodiment of the invention, the method includes using a lancing instrument having a sharpened end and a fluid channel extending from the sharpened end to a sensor attached to a proximal end of the lancing instrument, forcing the sharpened tip of the lancing element into the skin to a first predetermined depth, wherein the sharpened tip creates an incision in the skin surface and a wound below the skin surface and completely withdrawing the lancing element, including the sharpened tip. After completely withdrawing the sharpened tip from the incision, blood is drawn through the channel to the sensor.

In a method of lancing skin according to the present invention, the lancing element is inserted through an outer surface of the skin to obtain a sample of blood. In one embodiment of the invention, the method includes providing pressure on the skin in a region surrounding the incision site (i.e. the site where the incision is to be made). Then using a lancing instrument having a sharpened end and a fluid channel extending from the sharpened end to a sensor attached to a proximal end of the lancing instrument, forcing the sharpened tip of the lancing element into the skin to a first predetermined depth, wherein the sharpened tip creates an incision in the skin surface and a wound below the skin surface and completely withdrawing the lancing element, including the sharpened tip. After completely withdrawing the sharpened tip from the incision, and blood is drawn through the channel to the sensor.

In a method of lancing skin in accordance with the present invention, as set forth above, the method may further include providing a milking ring wherein the pressure in the region surrounding the incision site is exerted by the milking ring. The milking ring is positioned on the skin prior to the step of forcing the sharpened tip into the skin and may be maintained throughout the remainder of the procedure. In this embodiment of the invention, the milking ring provides a pressure sufficient to facilitate the flow of bodily fluids into the channel after the reinsertion of the lancing tip into the wound. In one embodiment of the invention, the milking ring provides a pressure in a range of approximately 0.5 to 1.5 pounds.

In a method of lancing skin in accordance with the present invention, as set forth above, the method may further include providing a milking ring wherein the pressure in the region surrounding the incision site is exerted by the milking ring. The milking ring is positioned on the skin prior to the step of forcing the sharpened tip into the skin and may be maintained throughout the remainder of the procedure. In this embodiment of the invention, the milking ring provides a pressure sufficient to facilitate the flow of bodily fluids into the channel after the reinsertion of the lancing tip into the wound. In one embodiment of the invention, the milking ring provides a pressure in a range of approximately 0.5 to 1.5 pounds. In a further embodiment of the present invention, the method may include positioning the milking ring against the skin for a predetermined period of time prior to launching the lancing element. In a further embodiment of the present invention, the predetermined period of time may be three seconds or more.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a lancing element and strip for use in a method according to the present invention.

FIG. 2 is a perspective view of the top layer of a lancing element and strip for use in a method according to the present invention.

FIG. 3A is a perspective view of a lancing element tip immediately before penetration of the surface of the skin.

FIG. 3B is a side view of the lancing element illustrated in FIG. 3A.

FIG. 3C is a perspective view of a lancing element as the tip of the lancing element begins to penetrate the surface of the skin.

FIG. 3D is a side view of the lancing element as the tip of the lancing element begins to penetrate the surface of the skin.

FIG. 3E is a perspective view of the lancing element as it reaches its full depth of penetration beneath the surface of the skin.

FIG. 3F is a side view of the lancing element as it reaches its full depth of penetration beneath the surface of the skin.

FIG. 3G is a perspective view of the lancing element after it is fully withdrawn from the skin.

FIG. 3H is a side view of the lancing element after it is fully withdrawn from the skin.

FIG. 3I is a perspective view of the lancing element as it re-enters the surface of the skin through the incision created during the first entry.

FIG. 3J is a side view of the lancing element as it re-enters the surface of the skin through the incision created during the first entry.

FIG. 3K is a perspective view showing the lancing element being used to draw blood from a forearm.

### DETAILED DESCRIPTION OF ILLUSTRATIVE EMBODIMENTS OF THE INVENTION

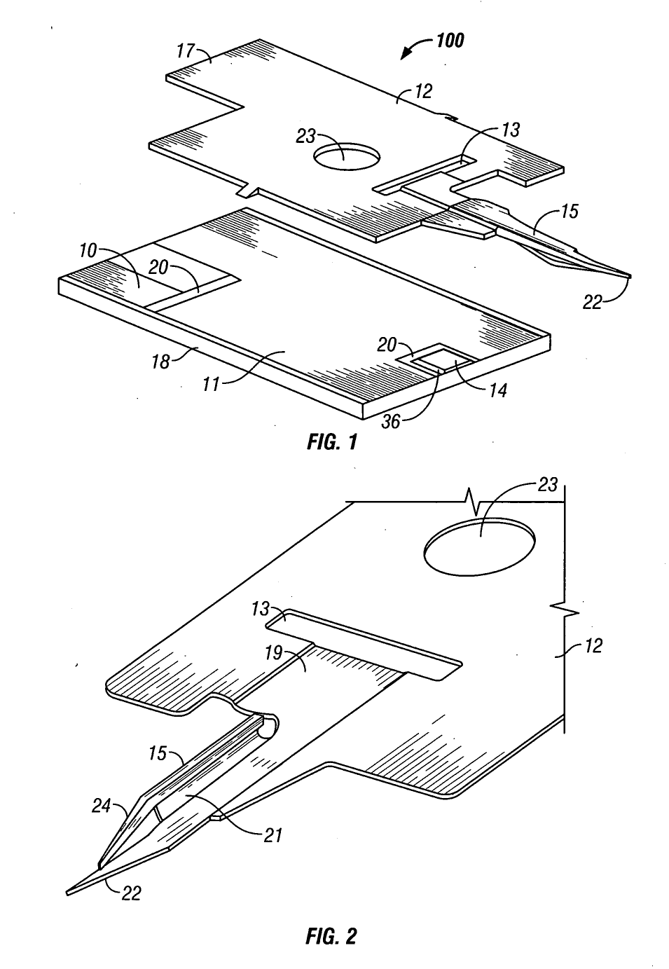

FIG. 1 is a perspective view of a lancing element and strip for use in a method according to the present invention. In FIG. 1, lancing element 15 is connected to sensor strip 100. Sensor strip 100 may be, for example, a glucose sensor strip which uses electrochemistry to measure the amount of glucose in a bodily fluid, such as, for example, blood or interstitial fluid. In FIG. 1, lancing element further includes lancing tip 22. Sensor strip 100 further includes first electrode contact 10, adhesive layer 11, conductive substrate 12, vent hole 13, analyte sensing layer 14, second electrode contact 17, insulating substrate 18, insulating layer 20, registration hole 23 and working electrode 36.

FIG. 2 is a perspective view of the top layer of a lancing element and strip for use in a method according to the present invention. In FIG. 2 top layer is formed of conductive substrate 12. In the embodiment illustrated in FIG. 2, conductive substrate 12 includes vent hole 13 and registration hole 23. In FIG. 2, lancing element includes lancing tip 22, channel tip 24 and fill channel 21.

One embodiment of a lancing element and sensor strip suitable for use in a method according to the present invention may be described with reference to FIGS. 1 and 2. In the embodiment illustrated in FIGS. 1 and 2, sensor strip 100 includes first electrode contact 10, wherein first electrode contact may be screen printed on an insulating substrate 18, and a second electrode contact 17, wherein said second electrode contact comprises a portion of conductive substrate 12 which is contiguous with top reference electrode 19 and lancing element 15.

In the embodiment of the lancing element and sensor strip illustrated in FIGS. 1 and 2, the orientation of said first electrode contact 10 and second electrode contact 17 are arranged such that an analyte measurement meter, such as, for example, a glucose meter (not shown) can establish electrical contact with sensor strip 100. In the illustrated embodiment, the electrodes are arranged on the same side of insulating substrate 18 to facilitate contact of both electrodes at the proximal end of sensor strip 100.

Sensor strip 100 is manufactured using adhesive layer 11 to attach insulating substrate 18 to conductive substrate 12. Adhesive layer 11 could be implemented in a number of ways, including using pressure sensitive material, heat activated material, or UV cured double sided adhesive material. Conductive substrate 12 may be, for example, a conductive substrate that is a sheet of electrically conductive material such as gold or plated stainless steel. The geometry of conductive substrate 12 may be formed by, for example, stamping process or photo-etching. In the embodiment illustrated in FIGS. 1 and 2, lancing element 15 may be manufactured as an integral part of conductive substrate 12. Vent hole 13, may be formed by, for example, punching through conductive layer 12. Vent hole 13 is used to facilitate the transport of bodily fluid up lancing element 15 and across analyte sensing layer 14. Registration hole 23 may be formed during the stamping process of making conductive substrate 12.

In one embodiment of the invention, analyte sensing layer 14 may be, for example, a glucose sensing layer, including an enzyme, a buffer, and a redox mediator. Analyte sensing layer 14 may preferably be deposited on top of working electrode 36. Where analyte sensing layer 14 is used to detect the presence and concentration of glucose in a bodily fluid, at least a portion of glucose sensing layer 14 dissolves in the bodily fluid and is used to convert the glucose concentration into an electrically measured parameter which is proportional to the glucose concentration in the sample.

In the embodiment illustrated in FIGS. 1 and 2, lancing element 15 has a proximal and distal end and the proximal end is integrated with the top reference electrode 19 and said distal end is integrated with a lancing tip 22 and channel tip 24. The lancing element is formed by the process of stamping or photo-etching a conductive material sheet and bending it to the geometry shown in FIG. 2. In one embodiment, lacing tip 22 and channel tip 24 are slightly offset by about 0.005 to 0.020″, the design of lancing element 15 is adapted to assist in improving skin separation. The geometry illustrated in FIGS. 1 and 2 may enhance fluid egress because it helps spread and open a skin wound. In the embodiment illustrated in FIGS. 1 and 2, the lancing element 15 is contiguous with the top reference electrode 19 and electrode contact 17.

In the embodiment of the invention illustrated in FIG. 2, lancing element 15 includes fill channel 21, wherein capillary fill channel 21 facilitates the flow of body fluid from the wound to the analyte sensing layer 14. Fill channel 21 may facilitate the flow of bodily fluids by, for example, wicking or capillary action. In the embodiment illustrated in FIGS. 1 and 2 fill channel 21 has an open geometry which facilitates the wicking of viscous samples and provides for simpler manufacturing techniques when compared with closed capillary channels.

In the embodiment of sensor strip 100 illustrated in FIG. 1, insulating substrate 18 consists of material such as polyester or ceramic on which a conductive material can be printed onto the insulating layer through silk-screening, sputtering, or electro-less deposition. Conductive material deposited on insulating substrate 18 forms first electrode contact 10 and working electrode 36. Insulating layer 20 may be, for example, screen printed to form a boundary for the electrode contact 10 and the bottom working electrode.

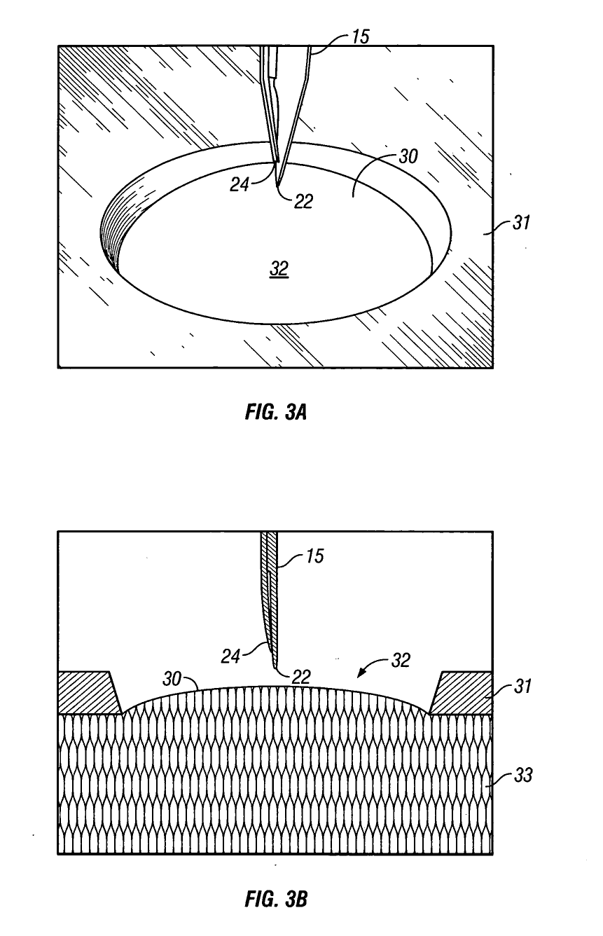

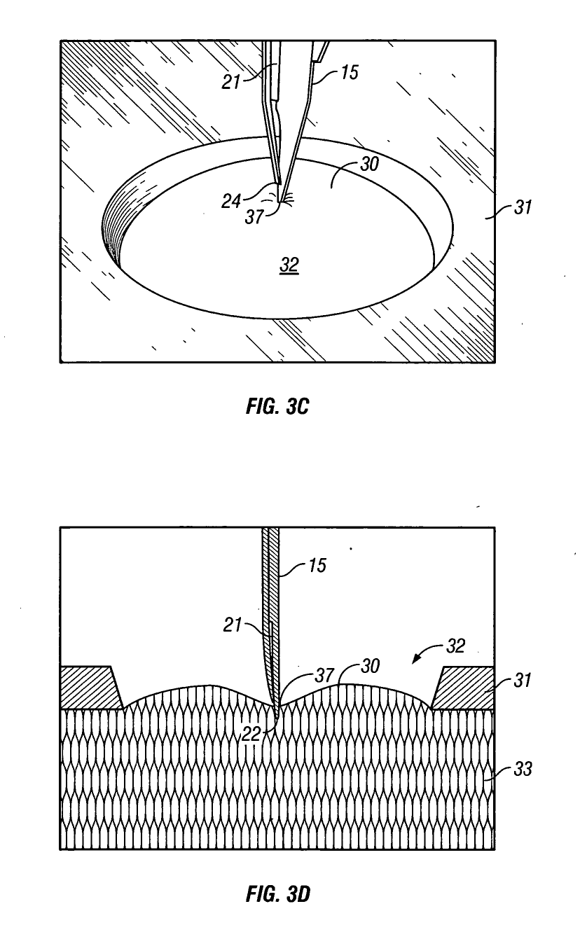

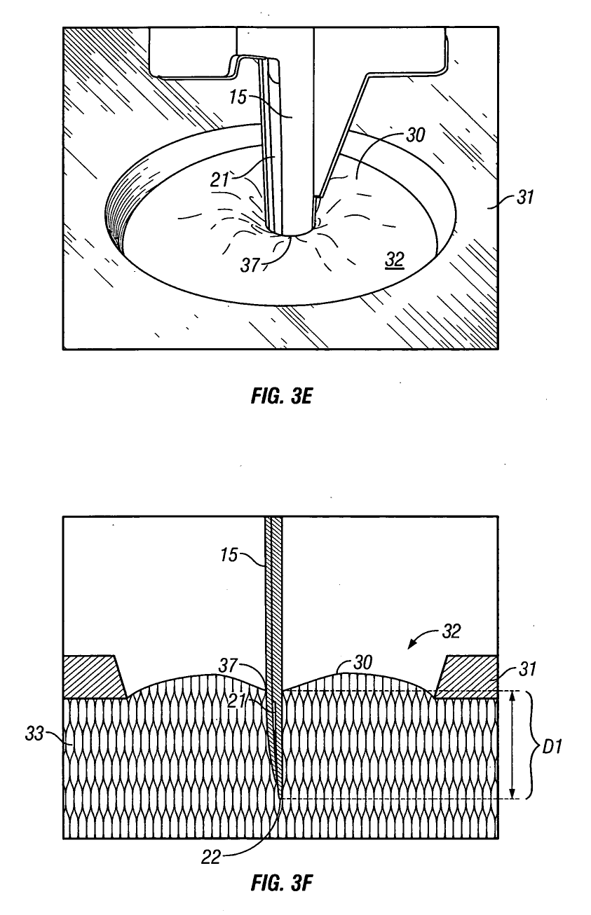

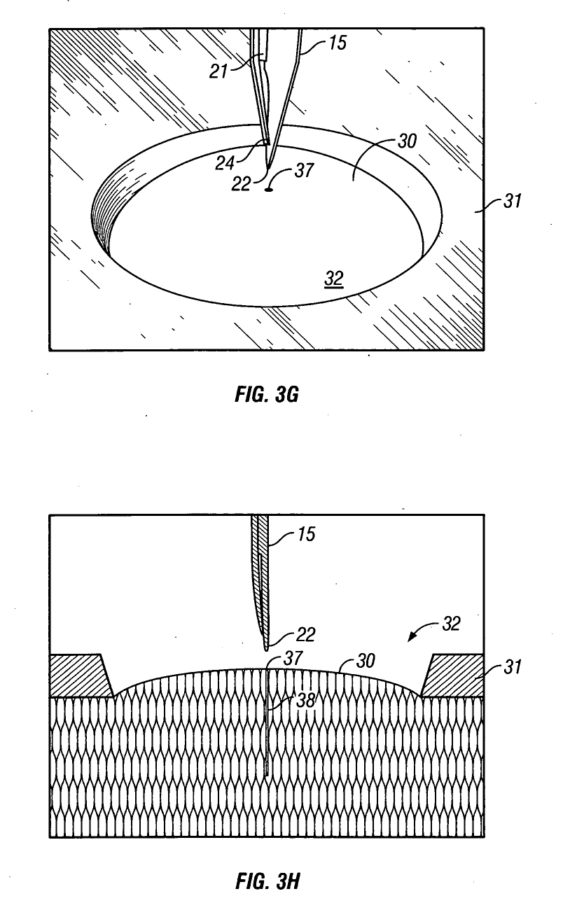

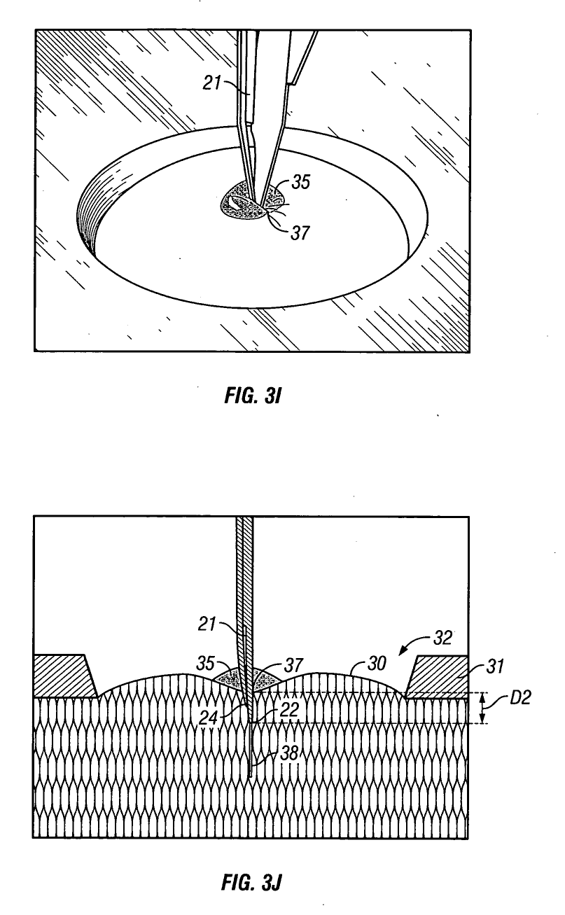

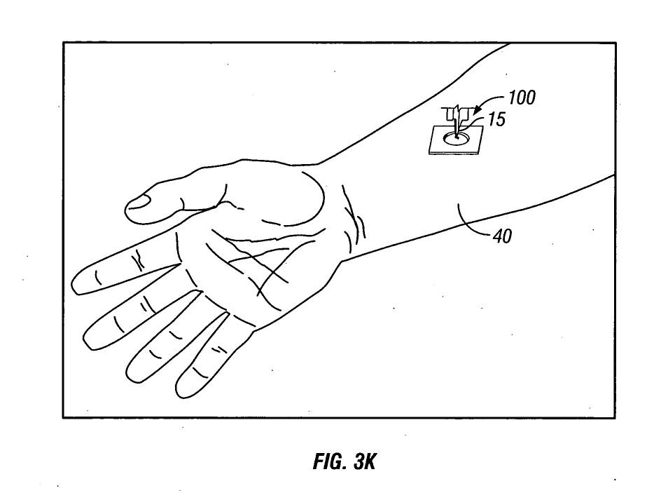

FIG. 3A is a perspective view of a lancing element tip immediately before penetration of the surface of the skin. More particularly, FIG. 3A is a perspective view of a lance 15 before lancing tip 22 penetrates skin surface 30. FIG. 3B is a side view of the lancing element illustrated in FIG. 3A. More particularly FIG. 3B is side view of lancing element 15 before lancing tip 22 penetrates skin surface 30. In FIGS. 3A and 3B, milking ring 31 is placed against skin surface 30, causing skin surface 30 to bulge into milking opening 32. Milking ring 32 may be, for example, a substrate with a hole drilled through it which could be, for example, a plastic such as polystyrene, polyethylene, polycarbonate, polyester, or the like. The diameter of opening 32 of said milking ring may be, for example, in the range of between 3.5 and 12 mm. In operation, the milking ring 31 may be applied with gentle pressure onto a fingertip, forearm, or other suitable site such that the skin surface 30 forms a raised mound within milking ring 31. In a one embodiment of a method according to the present invention, milking ring 31 is applied to skin surface 31 with a pressure of approximately 0.5 to 1.5 pounds of applied pressure. In one embodiment of a method according to the present invention, the use of a milking ring is intended to facilitate the collection of bodily fluids by applying a pressure around the incision site to provide a driving force for expressing fluid from the wound site.

FIG. 3C is a perspective view of a lancing element as the tip of the lancing element begins to penetrate the surface of the skin. As lancing tip 22 enters skin surface 30, deflecting skin surface 30 away from lancing tip 22 until skin surface 30 is punctured, forming an incision 37 in skin surface 30, enabling lancing element 15 to enter subcutaneous region 33. FIG. 3D is a side view of the lancing element as the tip of the lancing element begins to penetrate the surface of the skin. More particularly, FIG. 3D is a side view of lancing element 15 as lancing tip 22 enters incision 37 in skin surface 30.

FIG. 3E is a perspective view of the lancing element as it reaches its full depth of penetration beneath the surface of the skin. More particularly, FIG. 3E is a perspective view of lancing element 15 after lancing tip 22 has reached its full depth of penetration into subcutaneous region 33. FIG. 3F is a side view of the lancing element 15 as it reaches its full depth of penetration beneath the surface of the skin. More particularly, FIG. 3F is a side view of lancing element 15 after lancing tip 22 has reached its full depth of penetration into subcutaneous region 33. At full penetration, lancing tip 22 reaches a depth of D1. The actual value of D1 for a particular application will depend upon a number of factors, including the bodily fluid being extracted. For example, if the bodily fluid being extracted is blood, the depth D1 will be greater than if the bodily fluid being extracted is interstitial fluid (i.e. ISF). In one embodiment of the present invention, penetration depth D1 may be, for example, in the range of 0.25 to 1.5 mm deep.

In a method according to the present invention, insertion of lancing element 15 through skin surface 30 creates an incision 37 in addition to severing subcutaneous tissue and capillaries and providing fill channel 21 with access to the bodily fluid to be sampled, whether blood or interstitial fluid. Thus, with lancing element 15 positioned as shown in FIGS. 3E and 3F, bodily fluid will flow through fill channel 21 and into sensor strip 100. However, leaving lancing element 15 positioned as illustrated in FIGS. 3E and 3F will not provide an optimal flow of bodily fluid through fill channel 21. The reasons for the limited flow may include, for example, the blocking of lanced capillaries by the location of lancing element 15 which may, for example, prevent the capillaries or interstitial fluid from flowing freely and pooling in the wound created by lancing element 15.

FIG. 3G is a perspective view of the lancing element after it is fully withdrawn from the skin. More particularly, FIG. 3G is a perspective view of lancing element 15 after lancing tip 22 has been withdrawn completely from incision 37. FIG. 3H is a side view of the lancing element 15 after it is fully withdrawn from skin surface 30. In a method according to the present invention, fully withdrawing lancing element 15 from incision 37 creates an open the wound 38 below incision 37 which facilitates expression of bodily fluid into wound 38. By fully removing lancing element 15 from the wound in accordance with the method of the present invention, bodily fluids flow more readily into wound 38. If lancing element 15 is not completely removed from wound 38, blood and/or interstitial fluid flow may be impeded. One possible explanation for the limited blood flow is the possibility that the partially retracted lancing element 15 may still effectively block severed capillaries because of the resiliency of the skin. After the initial skin stretching during the penetration event, the skin might revert back to its initial position around lancing element 15. Thus, in the method according to the present invention it is important that lancing element 15 be fully removed from wound 38 after the initial penetration to allow bodily fluid to pool in wound 38.

FIG. 3I is a perspective view of the lancing element as it re-enters the surface of the skin through the incision created during the first entry. More particularly, FIG. 3I is a perspective view of lancing element 15 after lancing tip 22 has been re-inserted through incision 37 into wound 38. FIG. 3J is a side view of the lancing element as it re-enters the surface of the skin through the incision created during the first entry. More particularly, FIG. 3J is a side view of lancing element 15 positioned within wound 38 such that channel tip 24 is below skin surface 30. FIG. 3K is a perspective view showing the lancing element being used to draw blood from a forearm. More particularly, FIG. 3K is a perspective view of a lancing device 15 being used in a method according to the present invention to draw bodily fluids from a forearm 40 of a human being.

In a method of lancing skin in accordance with the present invention, as set forth above, the method may further include using milking ring 31 to exert the pressure in the region surrounding incision 37 exerted by milking ring 31. Milking ring 31 is positioned on the skin prior to the step of forcing the lancing tip 22 into the skin and may be maintained throughout the remainder of the procedure. In this embodiment of the invention, the milking ring 31 provides a pressure sufficient to facilitate the flow of bodily fluids into fill channel 21 after the reinsertion of lancing tip 22 into wound 38. In one embodiment of the invention, milking ring 31 provides a pressure in a range of approximately 0.5 to 1.5 pounds. In a further embodiment of the present invention, the method may include positioning the milking ring 31 against the skin for a predetermined period of time prior to launching the lancing element. In a further embodiment of the present invention, the predetermined period of time may be three seconds or more.

It will be recognized that equivalent structures may be substituted for the structures illustrated and described herein and that the described embodiment of the invention is not the only structure which may be employed to implement the claimed invention. As one example of an equivalent structure which may be used to implement the present invention, a lancing element may be used which does not include a channel tip, with the channel extending from the distal end of the lancing element to the working electrode. While preferred embodiments of the present invention have been shown and described herein, it will be obvious to those skilled in the art that such embodiments are provided by way of example only. Numerous variations, changes, and substitutions will now occur to hose skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of lancing skin through an outer surface of said skin to obtain a sample of blood, the method comprising the steps of:
providing a lancing instrument having a sharpened tip and a channel adjacent to said sharpened tip to a sensor attached to a proximal end of said lancing instrument;
forcing said sharpened tip into said skin to a first predetermined depth below said outer surface, wherein said sharpened tip creates an incision in said skin surface;
completely withdrawing said sharpened tip from said incision; and
drawing blood through said channel to said sensor.

2. A method of lancing skin as set forth in claim 1 wherein said first predetermined depth is in the range of approximately 0.25 to 1.5 mm.

3. A method of lancing skin as set forth in claim 1, further comprising the steps of providing pressure on said skin in a region surrounding said incision.

4. A method according to claim 3 wherein said pressure is exerted by a milking ring positioned on said skin prior to said step of forcing said sharpened tip into said skin.

5. A method according to claim 4 wherein said milking ring provides a pressure sufficient to facilitate the flow of bodily fluids into said channel.

6. A method according to claim 5 wherein said milking ring provides a pressure in a range of approximately 0.5 to 1.5 pounds.

7. A method of lancing skin as set forth in claim 3 wherein said pressure is applied for a predetermined time period prior to lancing said skin, said predetermined time period being approximately three seconds or more.
